(19) United States
(12) Patent Application Publication
(10) Pub. No.: US 2005/0222455 A1
(43) Pub. Date: Oct. 6, 2005

(54) Method for producing 3-hydroxy-2-methylbenzoic acid

(75) Inventors: Friedrich Muller-Hauck, Bendorf (DE); Johannes Scherer, Leverkusen (DE); Gerhard Schroeder, St. Michaelisdonn (DE); Horst Behre, Odenthal (DE)

Correspondence Address: Bayer Chemicals Corporation;Patent Department, 100 Bayer Road, Pittsburgh, PA, 15205-9741, US

(73) Assignee: Bayer Chemicals AG, Leverkusen (DE)

(21) Appl. No.: 10/507,875

(22) PCT Filed: Mar. 10, 2003
(86) PCT No.: PCT/EP03/02415

§ 371 Date: Sep. 15, 2004

(30) Foreign Application Priority Data
Mar. 22, 2002 (DE) 102 12 885.5

(51) Int. Cl. C07C 51/16; C07C 51/255

(52) U.S. Cl. 562/410

(57) ABSTRACT

The invention relates to a method for producing 3-hydroxy-2-methylbenzoic acid or solutions containing salts of 3-hydroxy-2-methylbenzoic acid, with naphthalene as the starting substance. Said method essentially comprises the following steps: sulfonation, reaction with alkalis at high temperatures and the expedient preparation of the resultant reaction mixture.

The invention relates to an improved process for preparing 3-hydroxy-2-methylbenzoic acid by heating naphthalene-1,3,5-trisulfonic acid or salts thereof in the presence of alkali metal hydroxides.

3-Hydroxy-2-methylbenzoic acid is a valuable intermediate in the preparation of pharmaceuticals, for example HIV protease inhibitors, and agrochemicals, for example insecticides (see, for example, U.S. Pat. No. 5,484,926 und EP-A 639 559).

According to Dean et al. (J. Chem. Soc., 1961, 2773), 3-hydroxy-2-methylbenzoic acid can be prepared by heating sodium hydrogen 3-aminonaphthalene-1,5-disulfonic acid with two weight equivalents of sodium hydroxide and water at a pressure of 40 bar to from 275 to 280° C., and, after the reaction mixture has been cooled, filtering and acidifying.

A similar process (DE 197 30 848 A) likewise starts from salts of 3-amino-naphthalene-1,5-disulfonic acid.

However, both methods have the disadvantage that both expensive aminonaphthalene derivatives and large amounts of alkalis have to be used as the starting material and cause a large amount of waste salts in the workup. In addition, a high pressure is established and ammonia is freed, which has to be removed.

According to DRP 91201, 3-hydroxy-2-methylbenzoic acid can also be prepared by heating naphthalene-1,3,5-trisulfonic acid to from 150 to 300° C. with two weight equivalents of sodium hydroxide, and, after the reaction mixture has been cooled, acidifying. A disadvantage of this process is that large amounts of alkalis have to be used and large amounts of sulfur dioxide are formed in the workup as a result of the use of the acid.

There is therefore a need to develop a process which, starting from readily available starting compounds, enables the preparation of solutions comprising salts of 3-hydroxy-2-methylbenzoic acid or 3-hydroxy-2-methylbenzoic acid in an advantageous manner.

A process has now been found for preparing 3-hydroxy-2-methylbenzoic acid or solutions comprising salts of 3-hydroxy-2-methylbenzoic acid, which is characterized in that
a) naphthalene-1,3,5-trisulfonic acid or salts thereof or mixtures thereof are heated in the presence of alkali metal hydroxide, water and optionally alkaline earth metal hydroxide,
b) the constituents which are insoluble in the resulting reaction mixture are removed, optionally after addition of water, and
c) soluble, undesired constituents are optionally removed from the reaction solution resulting from step b) and
d) acidification is optionally effected.

Naphthalene-1,3,5-trisulfonic acid can be prepared, for example, by initially reacting naphthalene with concentrated sulfuric acid or fuming sulfuric acid to give naphthalene-1,5-disulfonic acid and then converting it, without or after intermediate isolation (for example as the free acid or alkali metal salt), to a naphthalene-1,3,5-trisulfonic acid sulfation mixture (see, for example, Fierz-David, Grundlegende Reaktionen der Farbenchemie, Verlag Springer, Vienna 1952).

For example, what is known as the cold sulfation process with sulfur trioxide in a solvent can also be used to initially prepare naphthalene-1,5-disulfonic acid and this can be converted, without or after intermediate isolation (for example as the free acid or alkali metal salt), with further sulfur trioxide to a naphthalene-1,3,5-trisulfonic acid sulfation mixture (see, for example, EP-A 12 260 and DE-A 29 01 178).

Salts of naphthalene-1,3,5-trisulfonic acid are, for example and with preference, ammonium, alkali metal or alkaline earth metal salts.

Ammonium, alkali metal or alkaline earth metal salts of naphthalene-1,3,5-trisulfonic acid can be prepared from the isolated naphthalene-1,3,5-trisulfonic acid, for example, by adding ammonia or aqueous solutions thereof, alkali metal bases or alkaline earth metal salts.

In the context of the invention, ammonium, alkali metal and alkaline earth metal salts of naphthalene-1,3,5-trisulfonic acid are those naphthalene-1,3,5-trisulfonic acids in which one, two or three protons are each independently replaced by one equivalent of ammonium, of an alkali metal or a half equivalent of an alkaline earth metal. In the context of the invention, the terms mentioned likewise encompass any existing hydrates of such salts.

Surprisingly, alkali metal salts of naphthalene-1,3,5-trisulfonic acid can be prepared particularly advantageously by
i) reacting naphthalene with fuming sulfuric acid
ii) admixing the sulfation mixture resulting from 1), optionally after dilution with water, directly with an alkali metal base.

In a preferred embodiment for preparing alkali metal salts of naphthalene-1,3,5-trisulfonic acid, the procedure is, for example, to initially charge concentrated sulfuric acid, and add naphthalene and fuming sulfuric acid, and react the resulting reaction mixture, after dilution with water, with an alkali metal base.

In the context of the invention, concentrated sulfuric acid means, for example, sulfuric acid comprising from 90 to 100% by weight of H₂SO₄. In the context of the invention, fuming sulfuric acid is sulfuric acid which has a content of over 100% by weight based on H₂SO₄, i.e. contains free SO₃ and/or more highly condensed sulfuric acids, for example disulfuric acid H₂S₂O₇. Another common term for fuming sulfuric acid in the sense of the invention is oleum.

In the case of oleum, the specified content of free SO₃ (which also includes more highly condensed sulfuric acids) may be, for example, from 30 or 65% by weight.

Preference is given to using sufficient oleum that the molar ratio of free SO₃ to naphthalene is between 1.5:1 and 10:1, preferably between 2:1 and 5:1 and more preferably between 2.5:1 and 4:1.

The temperature in the addition may be, for example, from −20 to 70° C., preferably from 20 to 55° C.

The time for the addition may be, for example, between 10 min and 48 h, preferably from 2 h to 24 hours.

Subsequently, the resulting reaction mixture may optionally also be heated. The temperature may be, for example, between 55 and 150° C., preferably between 80 and 100° C.

Suitable alkali metal bases for step ii) are, for example, alkali metal carbonates, hydrogencarbonates and hydroxides which can be used in solid form or as aqueous solutions or dispersions.

The alkali metal bases used are preferably alkali metal hydroxides, of which still further preference is given to sodium hydroxide, potassium hydroxide or mixtures thereof, and very particular preference to sodium hydroxide.

The temperature of the reaction for step ii) may be, for example, from 0 to 100° C., preferably from 80 to 100° C.

The amount of the alkali metal hydroxide used may be, for example, from 2 to 10 times based on the molar ratio of the naphthalene used in step i), preferably from 2.8 to 3.5 times.

After workup in a manner known per se which may be effected, for example, by filtration and optionally washing and drying the precipitated solid, alkali metal salts of naphthalene-1,3,5-trisulfonic acid can be isolated and either stored or preferably reacted further.

Useful alkali metal salts of naphthalene-1,3,5-trisulfonic acid are, for example, the tri(alkali metal) naphthalene-1,3,5-trisulfonates, for example trisodium naphthalene-1,3,5-trisulfonate and tripotassium naphthalene-1,3,5-trisulfonate.

Tri(alkali metal) naphthalene-1,3,5-trisulfonates feature high storage stability (for example without caking) and ease of handling.

Optionally, the alkali metal salts of naphthalene-1,3,5-trisulfonic acid obtained and isolated in step ii) may be still further purified, for example, by recrystallization, although this is not necessary for the use in step a) of the process according to the invention.

It is possible to prepare alkaline earth metal salts of naphthalene-1,3,5-trisulfonic acid, for example, by dissolving the alkali metal salts of naphthalene-1,3,5-trisulfonic acid or the free acid itself in water and precipitating the desired products by adding solutions of alkaline earth metal salts, for example calcium chloride.

Step a) of the process according to the invention comprises the heating of salts of naphthalene-1,3,5-trisulfonic acid or of naphthalene-1,3,5-trisulfonic acid or mixtures thereof in the presence of alkali metal hydroxide, water and optionally alkaline earth metal hydroxide.

Since, according to Erdmann, Nieszytka (Liebigs Ann. Chem., 361, 171, 1908), free naphthalene-1,3,5-trisulfonic acid is hygroscopic, tends to bathe together and is therefore more difficult to handle, preference is given to using, in step a), ammonium, alkali metal or alkaline earth metal salts of naphthalene-1,3,5-trisulfonic acid, preferably the alkali metal salts of naphthalene-1,3,5-trisulfonic acid. Particular preference is given to using trisodium naphthalene-1,3,5-trisulfonate, tripotassium naphthalene-1,3,5-trisulfonate, of which trisodium naphthalene-1,3,5-trisulfonate is even more preferred.

The alkali metal hydroxide used may be, for example and with preference, sodium hydroxide and potassium hydroxide, for example as a solid or in the form of aqueous solutions or mixtures thereof.

The molar ratio of alkali metal hydroxide used to naphthalene-1,3,5-trisulfonic acid or its alkali metal and alkaline earth metal salts may be, for example, from 8 to 14 times, preferably from 10 to 13 times.

Preferably, alkaline earth metal hydroxide may additionally also be added. Suitable alkaline earth metal hydroxides are, for example and with preference, magnesium hydroxide and calcium hydroxide, of which calcium hydroxide is still more preferred.

The molar ratio of alkaline earth metal hydroxide used to naphthalene-1,3,5-trisulfonic acid or its salts may be, for example, from 0.1 to 8 times, preferably from 2 to 5 times and more preferably from 2.5 to 3.0 times.

The weight ratio of water to the sum of alkali metal hydroxide and any alkaline earth metal hydroxide added may be, for example, from 1:1.4 to 1:6.0, preferably a ratio of from 1:1.7 to 1:5.0.

The pressure in the reaction may be, for example, from 1 to 200 bar, preferably from 1 to 60 bar and more preferably that pressure that is established by heating the reaction mixture to the reaction temperature in a closed vessel starting from ambient pressure.

The closed vessel used may be, for example, an autoclave which may be made, for example, of nickel or other materials resistant toward alkalis.

The temperature of the reaction may be, for example, from 250 to 320° C., preferably from 270 to 320° C., more preferably from 305 to 320° C.

The reaction time may be, for example, from 2 to 12 hours, preferably from 3 to 10 hours, more preferably from 4 to 8 hours.

In a very particularly preferred embodiment, the reaction temperature is from 270 to 320° C. and the reaction time from 4 to 8 hours.

Subsequently, step b) is carried out, the removal of constituents which are insoluble in the resulting reaction mixture, optionally after addition of water.

Without any claim to completeness, the insoluble constituents comprise inorganic sulfites, sulfates and/or organic reaction by-products.

After the reaction mixture has been cooled, preference is given to adding water, which may be effected, for example, by pouring the resulting reaction mixture into water or onto ice.

Constituents which are insoluble in the resulting reaction mixture may be removed, for example and with preference, by filtration, centrifugation, sedimentation and decantation, optionally in the presence of assistants.

Assistants may be, for example, kieselguhrs, for example Celite®, activated carbon, for example Norite®, Acticarben, bleaching earth, montmorillonite or animal charcoal.

Preference is given to filtration, particular preference to filtration in the presence of assistants, preferably activated carbon.

In this way, a solution comprising salts of 3-hydroxy-2-methylbenzoic acid is obtained and can optionally be subjected to step c).

In a preferred embodiment of the process according to the invention, soluble, undesired constituents, for example discolorations or organic by-products, can be at least partly removed in step c).

This step may be carried out, for example, at a pH from above 3.5 to 14.

In a preferred embodiment, the procedure is, for example, to initially acidify to a pH of from 3.5 to 12, preferably from 5 to 10.5.

Suitable for the acidification are, for example, acidic salts, for example ammonium chloride, or acids, for example inorganic or organic acids, for example carboxylic acids or sulfonic acids. Preference is given to using inorganic acids, for example sulfuric acid, hydrochloric acid, phosphoric acid, nitric acid and hydrobromic acid or mixtures thereof, of which still further preference is given to sulfuric acid and hydrochloric acid. Very particular preference is given to sulfuric acid.

Subsequently, extraction may be effected with an organic solvent. Suitable organic solvents are, for example, esters such as ethyl acetate and butyl acetate, aliphatic or aromatic, optionally halogenated hydrocarbons, for example benzine, benzene, toluene, xylenes, chlorobenzene, dichlorobenzenes, isopropylbenzene, petroleum ether, hexane, heptane, octane, isooctane, cyclohexane, methylcyclohexane, dichloromethane, chloroform or carbon tetrachloride; ethers such as diethyl ether or diisopropyl ether; ketones such as 2-butanone or methyl isobutyl ketone, or mixtures of such solvents.

In addition, the resulting reaction solution from b) may be freed, for example, of discolorations with a suitable adsorbent which is effected, for example and with preference, after the above extraction.

Suitable adsorbents are, for example, silica gels, aluminas, cellulose or activated carbon.

3-Hydroxy-2-methylbenzoic acid can be obtained by acidifying (step d) from the reaction solution resulting from b), optionally after carrying out step c).

In this step, the pH is brought, for example, to 3.5 or below, preferably to from 0 to 3.5, more preferably to from 0.3 to 2.5.

At this point, it should be pointed out that the reporting of the pH values is based on values at room temperature.

Suitable for the acidification are, for example, acidic salts or acids, for example inorganic or organic acids, e.g. carboxylic acids or sulfonic acids. Preference is given to inorganic acids, e.g. sulfuric acid, hydrochloric acid, phosphoric acid, nitric acid and hydrobromic acid, or mixtures thereof, of which still further preference is given to sulfuric acid and hydrochloric acid. Very particular preference is given to sulfuric acid.

In the inventive manner, 3-hydroxy-2-methylbenzoic acid is obtained and may in some cases at least partly precipitate directly out of the solution.

Suitable workup methods are, for example, extraction with suitable organic solvents, or the removal of precipitated 3-hydroxy-2-methylbenzoic acid by filtration, optionally in the presence of filtration assistants, centrifugation or sedimentation and decanting and optionally subsequent drying.

Suitable organic solvents for the extraction of 3-hydroxy-2-methylbenzoic acid are, for example, esters such as methyl acetate, ethyl acetate and n-butyl acetate, aliphatic or aromatic, optionally halogenated hydrocarbons, for example benzine, benzene, toluene, xylenes, chlorobenzene, dichlorobenzenes, isopropylbenzene, petroleum ether, hexane, heptane, octane, isooctane, cyclohexane, methylcyclohexane, dichloromethane, chloroform or carbon tetrachloride; ethers such as diethyl ether or diisopropyl ether; ketones such as 2-butanone or methyl isobutyl ketone, or mixtures of such solvents. The 3-hydroxy-2-methylbenzoic acid can be obtained after extraction in a manner known per se, for example by evaporating the solvent.

For further purification, 3-hydroxy-2-methylbenzoic acid may optionally be recrystallized or reprecipitated.

The 3-hydroxy-2-methylbenzoic acid prepared by the process according to the invention is suitable in particular for use in a process for preparing pharmaceuticals, for example HIV protease inhibitors and agrochemicals, for example crop protection agents and insecticides or intermediates thereof.

The 3-hydroxy-2-methylbenzoic acid prepared by the process according to the invention is also suitable for preparing 3-methoxy-2-methylbenzoic acid and 3-acetoxy-2-methylbenzoic acid.

The advantage of the process according to the invention is the use of inexpensive naphthalene or of salts of 1,3,5-naphthalenetrisulfonic acid, readily obtainable in the inventive manner, as a starting material, which leads high yields and purities of 3-hydroxy-2-methylbenzoic acid and the effective use of hydroxides, which leads to a reduced salt burden.

### EXAMPLES

### Example 1

Sulfonation of Naphthalene

A flask is initially charged with 3961 g of sulfuric acid (100%). To this are metered by adding within 15 h 1844 g of naphthalene and 5613 g of oleum (65%) simultaneously in such a way that the temperature remains between 20 and 55° C. On completion of addition, the mixture is heated to 92° C. and stirred at this temperature for a further 6 h. 11355 g of reaction mixture are obtained.

### Example 2

Preparation of Trisodium naphthalene-1,3,5-trisulfonate (Method I)

In a flask, 3602 g of the reaction mixture from Example 1 are diluted with 1207 g of distilled water. To this are added at from 90 to 100° C. 1095 g of sodium hydroxide solution (50%) and the mixture is allowed to cool to 40° C. The solid which precipitates out is filtered off and washed with 1000 g of sulfuric acid (10%). 1651 g of a yellowish-white, finely crystalline, water-moist solid (content: 62.0% trisodium 1,3,5-naphthalenetrisulfonate, 2.2% other trisodium trisulfonates) are obtained. This corresponds to an isolated yield of 51.6% of theory.

### Example 3

Preparation of Trisodium naphthalene-1,3,5-trisulfonate (Method II)

In a flask, 3602 g of the reaction mixture from Example 1 are diluted with 2268 g of washing water from Example 1 and 148 g of distilled water. To this are added at from 90 to 100° C. 1096 g of sodium hydroxide solution (50%) and the mixture is allowed to cool to 40° C. The solid which precipitates out is filtered off and washed first with 396 g of sulfuric acid (10%) and then with 500 g of sulfuric acid (5%) which has been saturated with trisodium 1,3,5-naphthalenetrisulfonate (content: 24.3%). 2329 g of a grayish-white, finely crystalline, water-moist solid (content: 69.2% trisodium 1,3,5-naphthalenetrisulfonate, 1.4% other trisodium trisulfonates) are obtained. This corresponds to an isolated yield of 81.2% of theory.

### Example 4

### Preparation of Trisodium naphthalene-1,3,5-trisulfonate (Method III)

In a flask, 3640 g of the reaction mixture from Example 1 are diluted with 1219 g of distilled water. To this are added at from 90 to 100° C. 1107 g of sodium hydroxide solution (50%) and the mixture is allowed to cool to 50° C. The solid which precipitates out is filtered off and washed first with 500 g of sulfuric acid (5%) which has been saturated with trisodium 1,3,5-naphthalenetrisulfonate (content: 24.3%). 1768 g of a white, finely crystalline, water-moist solid (content: 67.9% trisodium 1,3,5-naphthalenetrisulfonate, 2.1% other trisodium trisulfonates) are obtained. This corresponds to an isolated yield of 50.9% of theory.

### Example 5

### Reaction with Alkalis (Method I)

In a nickel autoclave are stirred into a mixture of 232.0 g of aqueous sodium hydroxide solution (45%, 2.6 mol), 194.0 g of sodium hydroxide (99%, 4.8 mol) and 300.0 g of trisodium 1,3,5-naphthalenetrisulfonate (83.6%, 2.6% other trisulfonates, 13% water, 0.58 mol of reactant, 0.60 mol of trisulfonates), in such a way that an efficiently stirrable, slurrylike suspension is formed. The autoclave is sealed, heated to 190° C. without stirring and heated at this temperature for 30 minutes. Afterward, the stirrer is switched on and the autoclave heated to 280° C. The internal pressure (autogenous pressure) rises to 18.4 bar. The mixture is stirred at the reaction temperature for a further 6 h and then cooled to 90° C. At this temperature, 200.0 g of water are pumped in and the mixture is then cooled to room temperature. The resulting suspension is filtered off with suction and washed with 442.6 g of water. 176.1 g of a brownish-white, finely divided solid and 1061.2 g of a dark brown mother liquor (3-hydroxy-2-methylbenzoic acid product content approx. 6.6%) are obtained. This corresponds to a yield of 79.5% of theory.

### Example 6

### Reaction with Alkalis (Method II)

In a nickel autoclave, 300.0 g of trisodium 1,3,5-naphthalenetrisulfonate (83.6%, 2.6% other trisulfonates, 13% water, 0.58 mol of reactant, 0.60 mol of trisulfonates) are stirred into a mixture of 356.0 g of aqueous sodium hydroxide solution (45%, 4.0 mol), 126.0 g of sodium hydroxide (99%, 3.1 mol) and 127.0 g of calcium hydroxide (99%, 1.7 mol), in such a way that an efficiently stirrable, slurrylike suspension is formed. The autoclave is sealed, heated without stirring to 190° C. and heated at this temperature for 30 minutes. Afterward, the stirrer is switched on and the mixture is heated to 280° C. The internal pressure (autogenous pressure) rises to 29.3 bar. The mixture is stirred at the reaction temperature for a further 15 h and then cooled to 90° C. At this temperature, 200.0 g of water are pumped in and then the mixture is cooled to room temperature. The resulting suspension is filtered off with suction and washed with 500.0 g of water. 299.6 g of a brownish-white, finely divided solid (product content approx. 0.3%) and 1287.5 g of a dark brown mother liquor (product content 5.25%) are obtained. This corresponds to a yield of 76.9% of theory.

### Example 7

### Reaction with Alkalis (Method III)

In a nickel autoclave, 300.0 g of trisodium 1,3,5-naphthalenetrisulfonate (83.6%, 2.6% other trisulfonates, 13% water, 0.58 mol of reactant, 0.60 mol of trisulfonates) are stirred into a mixture of 356.0 g of aqueous sodium hydroxide solution (45%, 4.0 mol), 126.0 g of sodium hydroxide (99%, 3.1 mol) and 127.0 g of calcium hydroxide (99%, 1.7 mol), in such a way that an efficiently stirrable, slurrylike suspension is formed. The autoclave is sealed, heated without stirring to 190° C. and heated at this temperature for 30 minutes. Afterward, the stirrer is switched on and the mixture is heated to 310° C. The internal pressure (autogenous pressure) rises to 50 bar. The mixture is stirred at the reaction temperature for a further 5 h and then cooled to 90° C. At this temperature, 200.0 g of water are pumped in and then the mixture is cooled to room temperature. The resulting suspension is filtered off with suction and washed with 548.3 g of water. 336.1 g of a brownish-white, finely divided solid (product content approx. 0.11%) and 1310.3 g of a dark brown mother liquor (product content 5.22%) are obtained. This corresponds to a yield of 77.8% of theory.

### Example 8

Workup (Method I)

A flask is initially charged with 500.0 g of mother liquor from Example 7 and adjusted using 108.0 g of sulfuric acid (100%) to a pH of 7.4, in the course of which the reaction mixture is allowed to heat to 80° C. 5.0 g of activated carbon are added and the mixture is heated to reflux. The reaction solution is clarified and washed with 250.0 g of water. The clarified reaction solution is heated to 95° C. and adjusted to pH 0.4 by metering in 129.5 g of sulfuric acid (100%). The mixture is heated at 90° C. for 4 h, in the course of which a vigorous nitrogen stream is allowed to bubble through the solution. The mixture is then cooled to 45° C. within 2 h. The product precipitates out as a white precipitate. The product is filtered off with suction on a glass suction filter and washed with 340.0 g of water. The precipitate is dried at 60° C. for 16 h in a vacuum drying cabinet. 903.6 g of mother liquor (0.3% 3-hydroxy-2-methylbenzoic acid) and 369.1 g of wash liquor (1.5% 3-hydroxy-2-methylbenzoic acid) and 25.4 g of 3-hydroxy-2-methylbenzoic acid (content: 95.6%) are obtained. This corresponds to an isolated yield of 74.5% of theory.

### Example 9

Workup (Method II)

A flask is initially charged with 500.0 g of mother liquor from Example 7 (product content approx. 6.6%) and adjusted using 108.0 g of sulfuric acid (100%) to a pH of 7.4, in the course of which the reaction mixture is allowed to heat to 80° C. 5.0 g of activated carbon are added and the mixture is heated to reflux. The reaction solution is clarified and washed with 250.0 g of water. The clarified reaction solution is heated to 95° C. and adjusted to pH 0.4 by metering in 123.9 g of sulfuric acid (100%). The mixture is heated at 90° C. for 4 h, in the course of which a vigorous nitrogen stream is allowed to bubble through the solution. The mixture is then cooled to 45° C. within 2 h. The product precipitates out as a white precipitate. The product is filtered off with suction on a glass suction filter and washed with 400.0 g of aqueous solution of 3-hydroxy-2-methylbenzoic acid (1.67% 3-hydroxy-2-methylbenzoic acid). The precipitate is dried at 60° C. for 16 h in a vacuum drying cabinet. 925.7 g of mother liquor (0.3% 3-hydroxy-2-methylbenzoic acid) and 404.1 g of wash liquor (1.8% 3-hydroxy-2-methylbenzoic acid) and 28.8 g of 3-hydroxy-2-methylbenzoic acid (3-hydroxy-2-methylbenzoic acid, content: 95.6%) are obtained. This corresponds to an isolated yield of 84.4% of theory.

### Example 10

Workup (Method III)

A flask is initially charged with 930.0 g of mother liquor from Example 8 (product content approx. 5.25%) and adjusted using 150.6 g of sulfuric acid (100%) to a pH of 7.2, in the course of which the reaction mixture is allowed to heat to 80° C. 8.0 g of activated carbon are added and the mixture is heated to reflux. The reaction solution is clarified and washed with 100.0 g of water. The clarified reaction solution is heated to 95° C. and adjusted to pH 0.5 by metering in 199.2 g of sulfuric acid (100%). The mixture is heated at 90° C. for 4 h, in the course of which a vigorous nitrogen stream is allowed to bubble through the solution. The mixture is then cooled to 45° C. within 2 h. The product precipitates out as a white precipitate. The product is filtered off with suction on a glass suction filter and washed with 350.0 g of water. The precipitate is dried at 60° C. for 16 h in a vacuum drying cabinet. 1170.0 g of mother liquor (0.2% 3-hydroxy-2-methylbenzoic acid), 391.2 g of wash liquor (1.1% 3-hydroxy-2-methylbenzoic acid) and 42.5 g of 3-hydroxy-2-methylbenzoic acid (3-hydroxy-2-methylbenzoic acid, content: 95.9%) are obtained. This corresponds to an isolated yield of 83.5% of theory.

### Example 11

Workup (Method IV)

A flask is initially charged with 655.0 g of mother liquor from Example 9 (product content approx. 5.25%) and adjusted using 107.8 g of sulfuric acid (100%) to a pH of 7.1, in the course of which the reaction mixture is allowed to heat to 80° C. 5.0 g of activated carbon are added and the mixture is heated to reflux. The reaction solution is clarified and washed with 150.0 g of water. The clarified reaction solution is heated to 95° C. and adjusted to pH 0.4 by metering in 117.2 g of sulfuric acid (100%). The mixture is heated at 90° C. for 4 h, in the course of which a vigorous nitrogen stream is allowed to bubble through the solution. The mixture is then cooled to 45° C. within 2 h. The product precipitates out as a white precipitate. The product is filtered off with suction on a glass suction filter and washed with 300.0 g of aqueous solution of 3-hydroxy-2-methylbenzoic acid (1.7% 3-hydroxy-2-methylbenzoic acid).

The precipitate is dried at 60° C. for 16 h in a vacuum drying cabinet. 914.7 g of mother liquor (0.2% 3-hydroxy-2-methylbenzoic acid), 328.4 g of wash liquor (1.7% 3-hydroxy-2-methylbenzoic acid) and 31.8 g of 3-hydroxy-2-methylbenzoic acid (3-hydroxy-2-methylbenzoic acid, content: 95.7%) are obtained. This corresponds to an isolated yield of 89.0% of theory.

1. A process for preparing 3-hydroxy-2-methylbenzoic acid or solutions comprising salts of 3-hydroxy-2-methylbenzoic acid,
a) comprising heating naphthalene-1,3,5-trisulfonic acid or salts thereof or mixtures thereof in the presence of alkali metal hydroxide and water, and
b) removing the constituents which are insoluble in the resulting reaction mixture.

2. The process of claim 1, wherein soluble, undesired constituents are removed from the reaction solution resulting from b).

3. The process of claim 2, wherein undesired constituents which are soluble in the reaction solution by removed by extraction at a pH of from above 3.5 to 14.

4. The process of of claims 1, wherein the reaction solution resulting from b) is acidified with an acidic salt or an acid.

5. The process of claim 4, wherein acidification is effected to a pH of 3.5 or below.

6. The process of claims 1, wherein alkali metal salts of naphthalene-1,3,5-trisulfonic acid are used in step a).

7. The process of claim 6, in that wherein the alkali metal salts of naphthalene-1,3,5-trisulfonic acid are prepared by
i) reacting naphthalene with fuming sulfuric acid and
ii) reacting the resulting sulfation mixture with an alkali metal base.

8. The process of claim 7, wherein, in step i), concentrated sulfuric acid is initially charged and naphthalene and fuming sulfuric acid are added.

9. The process of claim 7, wherein, in step i), the molar ratio of free SO₃ from the fuming sulfuric acid to naphthalene is between 1.5:1 and 10:1.

10. The process of claim 7, wherein, in step ii), the resulting sulfation mixture from step i) is reacted with alkali metal hydroxides or aqueous solutions thereof.

11. The process of claim 1, wherein, in step a), alkali metal salts of naphthalene-1,3,5-trisulfonic acid are used.

12. The process of claim 1, wherein, in step a), trisodium naphthalene-1,3,5-trisulfonate is used.

13. The process of claim 1, wherein step a) is carried out in the presence of alkaline earth metal hydroxide.

14. The process of claim 13, wherein, in step a), the molar ratio of alkaline earth metal hydroxide used to naphthalene-1,3,5-trisulfonic acid or its alkali metal and alkaline earth metal salts is from 0.1 to 8 times.

15. The process of claim 13, wherein step a) is carried out in the presence of calcium hydroxide.

16. The process of one or more of claims 1, wherein, in step a), the molar ratio of alkali metal hydroxide used to naphthalene-1,3,5-trisulfonic acid or its alkali metal and alkaline earth metal salts is from 8 to 14 times, preferably from 10 to 13 times.

17. The process of claim 1, wherein, in step a), the weight ratio of water to the sum of alkali metal hydroxide and any alkaline earth metal hydroxide added is from 1:1.4 to 1:6.0.

18. The process of claim 1, wherein, in step a), the pressure in the reaction is from 1 to 200 bar.

19. The process of claim 1, wherein, in step a), the temperature of the reaction is from 250 to 320° C.

20. The process of claim 1, wherein, in step a), the reaction time is from 2 to 12 hours.

21. The process of claim 1, wherein, in step b), constituents which are insoluble in the reaction mixture are removed after addition of water.

22. The process of claim 1, wherein, in step b), constituents which are insoluble in the reaction mixture are removed by filtration.

23. A solution comprising salts of 3-hydroxy-2-methylbenzoic acid, obtainable by
a) heating naphthalene-1,3,5-trisulfonic acid or salts thereof or mixtures thereof in the presence of alkali metal hydroxide and water, and
b) removing the constituents which are insoluble in the resulting reaction mixture.

24. A method for preparing pharmaceuticals, agrochemicals or intermediates thereof with the solution according to claim 23.

25. The use of 3-hydroxy-2-methylbenzoic acid which has been prepared according to claim 1 for preparing pharmaceuticals, agrochemicals or intermediates thereof.

26. The use of 3-hydroxy-2-methylbenzoic acid which has been prepared according to of claim 1 for preparing insecticides.

27. The use of 3-hydroxy-2-methylbenzoic acid which has been prepared according to claim 1 for preparing 3-methoxy-2-methylbenzoic acid or 3-acetoxy-2-methylbenzoic acid.
